(19) United States
(12) Patent Application Publication
(10) Pub. No.: US 2019/0374243 A1
(43) Pub. Date: Dec. 12, 2019

(54) Device For Degreasing A Full-Thickness Skin Transplant

(71) Applicants: Steffan LOFF, Stuttgart (DE); MAJA-Maschinenfabrik Hermann Schill GmbH, Kehl-Goldscheuer (DE)

(72) Inventor: Steffan Loff, Stuttgart (DE)

(73) Assignee: MAJA-Maschinenfabrik Hermann Schill GmbH, Kehl-Goldscheuer (DE)

(21) Appl. No.: 16/341,576

(22) PCT Filed: Oct. 24, 2017
(86) PCT No.: PCT/EP2017/077198

§ 371 Date: Apr. 12, 2019

(30) Foreign Application Priority Data
Oct. 24, 2016 (DE) 10 2016 120 220.7

(51) Int. Cl. A61B 17/322

(52) U.S. Cl. CPC A61B 17/322; A61B 2017/00969

(57) ABSTRACT

The present invention relates to a device for degreasing a full-thickness skin transplant, comprising a cutting unit, which is designed and arranged to separate a layer of subcutaneous fatty tissue located on the full-thickness skin transplant. The device comprises a drawing roller which is arranged across a gap at a distance from a cutting edge of the cutting unit, and which is designed to deliver the full-thickness skin transplant to the cutting unit by rotating the drawing roller.

### BACKGROUND

Serious burns and scalds from a grade of “deep IIb” to “IV” regularly lead to scars independent of the quality of the first aid. Thick cicatricial bands often form in particular across joints and areas of greater movement, which can adversely affect the movement so much that the affected joint becomes unusable. Classic places for these serious complications of burns and scalds are the finger joints, the elbow joints, the shoulder joints, the knee joints and the neck. On the hand, a complete stiffening can occur in the case of deep wounds. On the shoulder and elbow joint, cicatricial contractions lead to an inability to use the affected arm. In common flexion contractures in the knee joints, affected patients cannot extend the leg and can no longer walk. After deep wounds on the neck, the feared sternomental contracture develops in which the chin is pulled toward the breast bone and fixed by cicatricial contraction. This leads to sometimes severe deformities of the face, ectropion of the eyes and even misalignment of the teeth.

Such deformities can be treated with surgical technology using appropriate reconstructive therapies. A distinction is made between so-called local flaps, free flaps and full-thickness skin transplants. Full-thickness skin transplants are the first choice among the corrective measures in the case of defects that cannot be covered by local flap reconstruction. With complete healing, they generally lead to a correction of the deformity.

Full-thickness skin transplants are made of autologous skin, meaning own-body skin, with epidermal structures such as hair follicles and sweat glands (the dermis in its full thickness). They have a thickness of 0.8-1.2 mm.

Full-thickness transplants are obtained if one removes a skin area of the required size at location with redundant fatty tissue. Typical places are the stomach fold, the gluteal grooves and the wrist grooves. In the removal, a spindle-shaped skin incision and excision of the skin area along with the fatty tissue lying below it is conducted. The removal is sized large enough that the resulting defect can be closed again via sutures after mobilization of the wound margins. The maximum size in the case of a stomach skin removal, for example, is limited in length by the distance between the two iliac crests and in width by the distance of the wound margins that just now can still be closed. In the case of large full-thickness skin transplants, this size can reach 30 cm×15 cm.

The desired full-thickness skin transplant is generally marked in the removal area using a stencil and then removed, including the subcutaneous fatty tissue. Before the full-thickness skin transplant can be placed on the site to be treated, the receiving region, and sutured into the wound margins, it must be completely degreased, meaning the subcutaneous fat layer must be removed as much as possible.

After the suturing in the receiving region, the full-thickness skin transplant is temporarily completely separated from the blood circulation. The full-thickness skin transplant is temporarily nourished only by diffusion from the tissue located below it. After approx. 5-7 days, new capillary buds arise in the full-thickness skin transplant and a blood circulation forms.

A supplying of the full-thickness skin transplant via diffusion can only take place if the full-thickness skin transplant is sufficiently thin. If the subcutaneous fatty tissue is not or is not sufficiently removed, the full-thickness skin transplant is then too thick, cannot be supplied via diffusion and dies away.

For the already mentioned reason, the degreasing of the full-thickness skin transplant after the removal is crucial for the success of a transplant. It is especially advantageous here if small fatty regions are left at individual places distributed over the extent of the full-thickness transplant. After the transplant, they form seed cells for the formation of a new subcutaneous fatty tissue.

Traditionally, the subcutaneous fatty tissue is manually removed from the full-thickness skin transplant via a scalpel or a pair of shears. This process is very time-consuming, which leads to a long duration of the corresponding transplant surgery. On the one hand, this presents a health burden for the patient, and, on the other hand, this results in high costs for a surgery of this type because the degreasing of a full-thickness transplant must be carried out by an experienced surgeon.

It is the object of the present invention to provide a device via which the subcutaneous fatty tissue can quickly and reliably be removed from the full-thickness skin transplant as completely as possible. In other words, a device, via which the full-thickness skin transplant can quickly and reliably be degreased.

### SUMMARY OF THE INVENTION

This object is achieved with a device for degreasing a full-thickness skin transplant having a cutting unit for separating a subcutaneous fatty tissue located on the full-thickness skin transplant, in that the device has a drawing roller which is arranged across a gap at a distance from a cutting edge of the cutting unit, and which is designed to deliver the full-thickness skin transplant to the cutting unit by rotating the drawing roller.

During operation of the device, the full-thickness skin transplant with the subcutaneous fatty tissue layer located thereon is attached to the drawing roller. The drawing roller is then rotated. By rotation of the drawing roller, the full-thickness skin transplant is guided against a cutting edge of the cutting unit. The gap between the drawing roller and the cutting unit is designed in such a manner that the full-thickness skin transplant is guided between the drawing roller and the cutting unit into the gap by the rotation of the drawing roller, while the cutting unit separates the subcutaneous fatty tissue from the full-thickness skin transplant. The separated subcutaneous layer of fat is then located on the side of the cutting unit opposite the gap and can be removed and disposed of

It is preferable if the cutting unit here has a blade that comprises the cutting edge and is mounted on a blade holder, in particular releasably. The blade can be simply removed and exchanged or cleaned if the blade is releasably mounted.

The blade is preferably mounted in the blade holder without play, in particular releasably, preferably braced. The device hereby permits a particularly reproducible operating performance.

It is preferred if the blade holder is arranged without play with respect to the drawing roller. The blade holder is preferably mounted releasably on the rest of the device.

In the present context, a play-free mounting means, in particular, a rigid attachment in such a manner that the parts attached to each other cannot move relative to each other.

The drawing roller preferably has a surface facing the drawing roller with a curved design. The surface facing the blade holder drawing roller is preferably designed having a radius of curvature that is different, preferably larger, than the radius of curvature of the drawing roller. The blade holder is preferably designed in such a manner and arranged with respect to the drawing roller in such a manner that a secondary gap, which corresponds to the distance between the drawing roller and the surface of the blade holder that faces the drawing roller on a side of the blade holder close to the blade, is less than a distance A on a side of the blade holder facing away from the blade holder. This results in an advantageous transport behavior of the drawing roller.

The distance between the drawing roller and the surface of the blade holder facing the drawing roller is preferably designed in such a manner that it increases in the direction of transport.

The side of the blade holder close to the cutting edge is preferably spaced apart from the drawing roller by a secondary gap, wherein the secondary gap is larger than the gap by which the cutting edge is spaced apart from the drawing roller.

The blade is preferably arranged braced between the blade holder and a clamping element. The blade is preferably mounted releasably on the device via the bracing between the blade holder and the clamping element. As a result, a releasable and nevertheless play-free mounting of the blade can be realized.

The blade and the blade holder, as well as any clamping element, are preferably arranged parallel to an axis of rotation of the drawing roller.

The blade preferably has a flat design and a blade thickness of 0.4 mm to 1 mm, preferably 0.5 mm to 0.8 mm, in particular 0.6 mm and 0.75 mm.

The blade preferably has a projection over the blade holder such that the cutting edge is spaced between 2 mm and 6 mm, preferably between 3 mm and 5 mm, from the blade holder.

The cutting edge of the blade is preferably spaced at a cutting edge distance from the axis of rotation of the drawing roller and a closest section of the blade is spaced at a minimum distance from the axis of rotation of the drawing roller, wherein the cutting edge distance is greater than the minimum distance. As a result, the feeding of the full-thickness skin transplant into the gap is improved.

The blade preferably has a flat design and is arranged with respect to the drawing roller in such a manner that, looking along the axis of rotation of the drawing roller, a line running perpendicular to the axis of rotation cuts the blade at an angle of 90°, wherein the cutting edge is preferably spaced at least 0.5 mm, preferably 1 mm, in particular 1.5 mm from the intersection of the line with the blade.

The device can preferably be disassembled. As a result, the device in its disassembled form can easily be cleaned in an autoclave.

The device is preferably operated purely mechanically. The device in this embodiment therefore does not include, for example, an electric drive.

The device is preferably designed for manual operation. It is preferred that the drawing roller can be rotated via a hand crank. In this manner, the operator can precisely control and adjust the advance of the full-thickness skin transplant. The operation of the device is therefore particularly reliable and accidental damage to the full-thickness skin transplant can be prevented.

Alternatively, the drawing roller can be rotatably driven via a drive unit, wherein a rotational speed of the drawing roller can be changed via a control element during the operation of the device. This embodiment represents a driven—meaning not manually operated, or, in any case, not exclusively manually operated-embodiment. In this embodiment, a high reproducibility of the rotational speed of the drawing roller can be achieved. The driven embodiment may also be combined with the hand crank; for example, a switchover or also a combination between the two drive types is conceivable. For example, a motor can guarantee a basic speed of the drawing roller that can be temporarily increased manually by actuating the hand crank.

In addition, it is advantageous if the cutting edge of the cutting unit is arranged essentially parallel to the axis of rotation of the drawing roller, preferably wherein the gap by which the cutting edge is spaced apart from the drawing roller has a constant gap width over the extent of the drawing roller along the axis of rotation. An especially clean and precise separation of the subcutaneous layer of fat is thereby ensured.

It is preferred if the gap between the cutting edge of the cutting unit and the drawing roller has a gap width of 0.8-1.2 mm. The gap is thus designed in such a way that the degreased full-thickness skin transplant has a thickness of 0.8-1.2 mm. Consequently, a manual post-processing of the full-thickness skin transplant can be omitted.

The gap can preferably be adjusted incrementally.

It is advantageous if the drawing roller has rib-like or knobby protrusions on its active surface. These protrusions are advantageously arranged distributed across the entire surface of the drawing roller. The protrusions are preferably rib-shaped and run parallel to an axis of rotation of the drawing roller. In a preferred embodiment, the drawing roller has a diameter of at least 4 cm, in particular 5 cm, preferably at most 8 cm, in particular at most 7 cm, and between 50 and 90, in particular between 55 and 80, rib-like protrusions are arranged in each case evenly distributed over the active surface of the drawing roller. The distance between the individual protrusions in the circumferential direction is 1.5 mm to 3.5 mm and particularly preferably between 2.0 mm and 3.0 mm.

On the one hand, the indicated protrusions lead to an improved contact of the full-thickness skin transplant by the drawing roller and, on the other, it is thereby achieved that the subcutaneous layer of fat is not completely removed along the whole extent of the full-thickness skin transplant; rather, there remain small regions of subcutaneous fat on the full-thickness skin transplant. These small regions of subcutaneous fat can serve in the transplanted state for the formation of a new subcutaneous layer of fat.

The drawing roller can have protrusions that are spaced apart from each other via recesses. The recesses can be formed with an arcuate cross-section. The recesses can have a radius of curvature that is larger than the radius of curvature of the protrusions. The protrusions can be spaced further apart from each other in the circumferential direction via the recesses than they are extended in the circumferential direction.

It is also advantageous if a pressure roller is arranged on a side of the cutting edge opposite the drawing roller and is designed to press the full-thickness skin transplant along with the subcutaneous layer of fat located thereon against the drawing roller. The pressure roller is preferably designed to be flexible. As a result, a particularly precise passage of the full-thickness skin transplant between gap and drawing roller is ensured. The full-thickness skin transplant including the subcutaneous layer of fat is precisely positioned effectively between the drawing roller and the pressure roller and then guided against the cutting unit. The flexible design of the pressure roller makes it possible to compensate for variations in the thickness of the full-thickness skin transplant including the fatty tissue.

It is also preferred if the gap width between the cutting edge and the drawing roller is adjustable. That is, if a position of the cutting edge of the cutting unit is adjustable with respect to the drawing roller. As a result, the thickness of the full-thickness skin transplant after the separation of the subcutaneous layer of fat can be adjusted according to the need. In other words, the device is adjustable in such a manner that a portion of the subcutaneous layer of fat is maintained on the full-thickness skin transplant.

It is also advantageous if the device can be disassembled and the cutting unit and the drawing roller in any case sterilized, in particular autoclaved. On the one hand, the individual parts of the device are releasable from each other in a simple manner. An example for easily releasable connections are plug-in connections. In order for the individual parts to be sterilizable or autoclavable, they must be as flat as possible and be designed without inaccessible places or the like. For example, contamination can accumulate in undercuts or similar inaccessible places and cannot be removed using a normal sterilization process. Medical requirements can be addressed via the ability to be sterilized.

In a preferred embodiment, the drawing roller has in any case an at least partly rounded active surface; the drawing roller preferably has a rounded design over its entire active surface. The term “active surface” means the surface of the drawing roller that can come into contact with the full-thickness skin transplant. Rounded here means that the active surface in the rounded region or preferably across the whole active surface has no sharp edges. All edges on the active surface or on the rounded region of the active surface are preferably rounded with a radius of curvature of at least 0.25 mm, in particular 0.5 mm, preferably 1.0 mm,

It is also advantageous if the drawing roller has at least one suction opening on its active surface, preferably a plurality of suction openings that are distributed over the active surface that are/can be connected with a vacuum source. As a result, the full-thickness skin transplant can be suctioned onto the drawing roller via the suction opening using a vacuum if it is placed on the drawing roller. As a result, a secure and slip-proof contact can be ensured between the drawing roller and the full-thickness skin transplant.

It is advantageous if the blade is affixed to the device, in particular on the blade holder, without screws.

It is advantageous if the device can be disassembled in such a manner that its individual parts are designed without a gap. The individual parts are easy to clean as a result of this.

Some, preferably all, individual parts are preferably designed without undercuts.

It is advantageous if the drawing roller comprises stainless steel, or preferably is made of stainless steel.

A part of the present invention is also the use of a device according to one or more of the described embodiments for degreasing a full-thickness skin transplant.

A part of the present invention is also a method for degreasing a full-thickness skin transplant, wherein the method comprises the steps:
attaching the full-thickness skin transplant with the subcutaneous layer of fat located thereon to a drawing roller in such a manner that the full-thickness skin transplant faces the drawing roller and the subcutaneous layer of fat faces away from the drawing roller;
guiding the full-thickness skin transplant with the subcutaneous layer of fat located thereon against a cutting edge of a cutting unit via the rotation of the drawing roller, wherein a gap is designed between the drawing roller and the cutting unit in such a manner that the full-thickness skin transplant is guided between the drawing roller and the cutting unit into the gap by the rotation of the drawing roller while the cutting unit separates the subcutaneous layer of fat from the full-thickness skin transplant.

The rotation of the drawing roller in the method is preferably accomplished manually, in particular via a hand crank.

The method is preferably carried out using one of the devices described above. It is advantageous in the method if the individual aspects of the device described above are provided in the method.

It is particularly advantageous if in the method the cutting edge of the cutting unit is arranged essentially parallel to the axis of rotation of the drawing roller.

It is particularly advantageous in the method if the drawing roller has rib-like or knobby protrusions on its active surface. These protrusions are advantageously arranged distributed across the entire surface of the drawing roller. The protrusions are preferably rib-shaped and run parallel to the axis of rotation of the drawing roller.

The drawing roller can have in particular a geometrically textured surface or active surface.

### BRIEF DESCRIPTION OF THE DRAWINGS

Additional features, application possibilities and advantages of the invention arise from the following description of exemplary embodiments of the invention and are explained in reference to the drawing, wherein the features can be important for the invention individually as well as in different combinations without this being additionally explicitly indicated. Shown are:

FIG. 1a perspective view of the device;

FIG. 2a sectional view of the device;

FIG. 3 the device from FIGS. 1 and 2 during operation in the view from FIG. 2;

FIG. 4 an alternative embodiment in the view from FIGS. 2 and 3;

FIG. 5 a schematic detailed view of a drawing roller of an alternative embodiment;

FIG. 6 an alternative embodiment in different views;

FIG. 7a sectional view through the drawing roller of the alternative embodiment; and

FIG. 8 a partial region from FIG. 7 in an enlarged view; and

FIG. 9 a sectional view through a drawing roller of the alternative embodiment.

### DETAILED DESCRIPTION

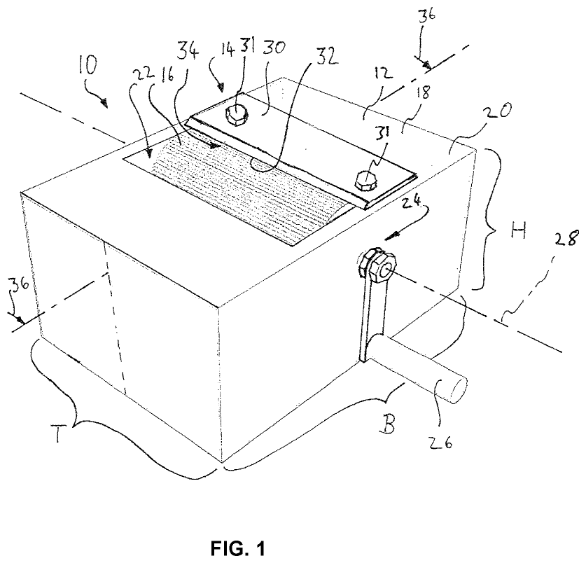

FIG. 1 shows a device 10 according to the invention for degreasing a full-thickness skin transplant. The device 10 comprises a base body 12, a cutting unit 14 and a drawing roller 16.

The base body 12 here essentially comprises a rectangular housing 18 made of stainless steel. In the present exemplary embodiment of the invention, the stainless steel housing has a width B of 30 cm in this example, a depth T of 20 cm in this example and a height H, also of 20 cm in this example. These measurements, however, are in no way to be understood as limiting, but only illustrate the presently described embodiment as an example. The present invention is also not limited to a base body 12 having the design of the present one, in particular to a housing 18 having the measurements indicated here. For example, the drawing roller 16 can also be provided in a device 10 completely without a housing 18.

The housing 18 has an opening 22 in a top side 20. Looking into the opening 22, the drawing roller 16 arranged in the interior of the housing 18 is visible.

The drawing roller 16 is rotatably connected to the base body 12 via a mounting 24. A hand crank 26 is attached to the outer side of the base body 12. The hand crank is connected to the drawing roller 16 in such a manner that the drawing roller 16 can be rotated around an axis of rotation 28 by operating the hand crank 26.

In the region of the opening 22, a blade 30 of the cutting unit 14 is arranged via two mounting and positioning elements 31 in such a manner that a cutting edge 32 of the blade 30 is arranged at a distance from the drawing roller 16 across a gap 34. The cutting edge 32 of the blade 30 runs parallel to the axis of rotation 28 of the drawing roller 16.

The blade 30 of the cutting unit is attached to the housing via the two mounting and positioning elements 31. In addition to the mounting of the blade 30, the mounting and positioning elements 31 also function as an adjustment mechanism for the cutting edge 32. The arrangement of the blade 30 or the gap 34 to the drawing roller 16 is adjustable and variable via the mounting and positioning elements 31.

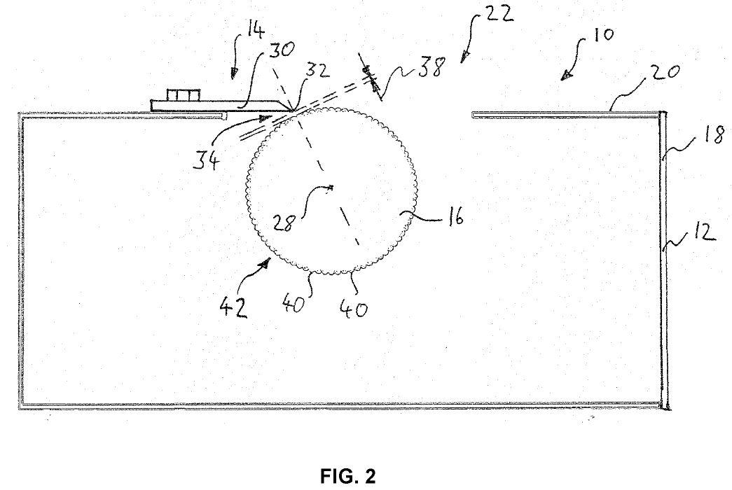

FIG. 2 shows the device 10 cut along the line II-II and seen in the direction of the arrow 36. The spaced arrangement of the drawing roller 16 with respect to the cutting edge 32 across the gap 34, in particular, can be clearly seen in FIG. 2. The gap 34 has a gap width 38 that here equals one millimeter. The gap width 38 corresponds to the distance of the cutting edge 32 from the surface of the drawing roller 16.

As can be seen in FIG. 2, the drawing roller 16 has a plurality of rib-like protrusions 40 that are arranged on an active surface 42 of the drawing roller 16. As can also be seen in FIG. 1, the rib-like protrusions 40 extend parallel to the axis of rotation 28 of the drawing roller 16. Only two of the protrusions 40 are provided with a reference character in FIG. 2. The active surface 42 of the drawing roller 16 here corresponds to the surface of the drawing roller 16 that runs parallel to the axis of rotation 28. Active surface 42 here means the part of the surface of the drawing roller 16 that is provided for the purpose of feeding the full-thickness skin transplant to the cutting unit 14.

The rib-shaped protrusions 40 in the present example each have a height of approx. 1 mm and are about 1 mm wide. Other dimensions are conceivable. The rib-shaped protrusions 40 in the present example have an essentially arcuate cross-section, wherein the corresponding circle in the present example has a diameter of 1 mm.

The gap width 38 is measured from the top side of the rib-like protrusions 40 to the cutting edge 32 of the blade 30.

The active surface 42 of the drawing roller 16 as a whole has a rounded design, meaning that the active surface 42 has no sharp edges. Each region that could be interpreted as an edge is rounded, wherein, for example, the rounding has a radius of at least one fourth mm, preferably a half mm.

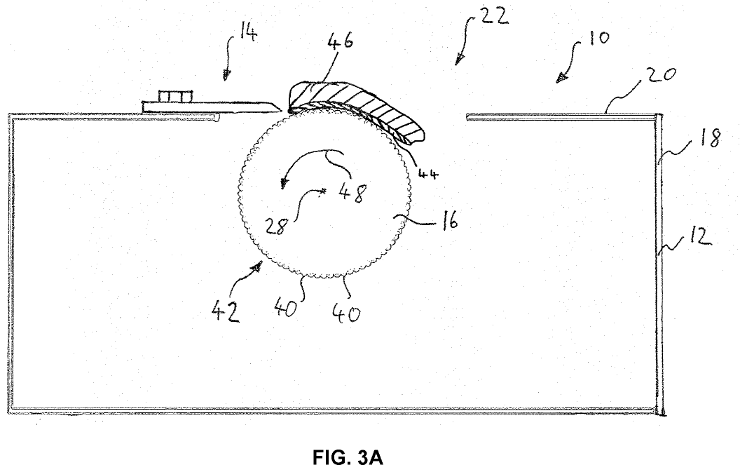

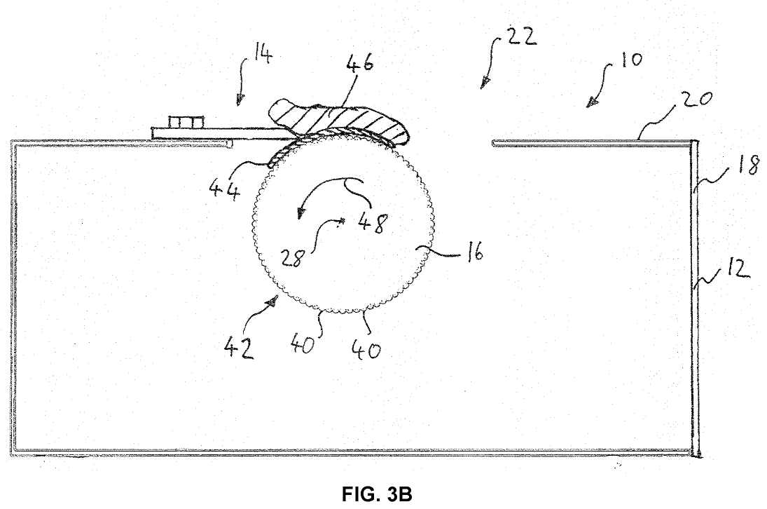

In FIGS. 3a and 3b, the device 10 from FIGS. 1 and 2 is shown in operation. Steps of the method according to the invention are also shown.

A full-thickness skin transplant 44 with the subcutaneous fatty tissue 46 located thereon is attached to the drawing roller 16 in FIG. 3a. The full-thickness skin transplant 44 thereby contacts the drawing roller 16 and the subcutaneous fatty tissue faces away from the drawing roller 16.

The drawing roller 16 is set into rotation via the hand crank 26, which is indicated by an arrow with the reference character 48. The full-thickness skin transplant 44 along with the subcutaneous layer of fat 46 located thereon is moved by the rotation from the position in FIG. 3a into the position in FIG. 3b and beyond.

The full-thickness skin transplant 44 with the subcutaneous layer of fat 46 located thereon is moved against the cutting edge 32 of the cutting unit 14 by the rotation of the drawing roller 16, wherein a gap 34 between the drawing roller 16 and the cutting unit 14 is formed in such a way that the full-thickness skin transplant 44 is guided by the rotation of the drawing roller 16 between the drawing roller 16 and the cutting unit 14 into the gap 34, while the cutting unit 14 separates the layer of fat 46 from the full-thickness skin transplant 44. This separation is illustrated in FIG. 3b.

The full-thickness skin transplant 44 with the subcutaneous fatty tissue 46 located thereon is guided by the rotation of the drawing roller 16 against the cutting edge 32 of the cutting unit 14. As a result, the subcutaneous fatty tissue 46 is separated from the full-thickness skin transplant 44. If the complete layer of the subcutaneous fatty tissue 46 is removed from the full-thickness skin transplant 44, the subcutaneous layer of fat 46 located thereon can thus be removed and disposed of. The full-thickness skin transplant 44 can also be removed and used.

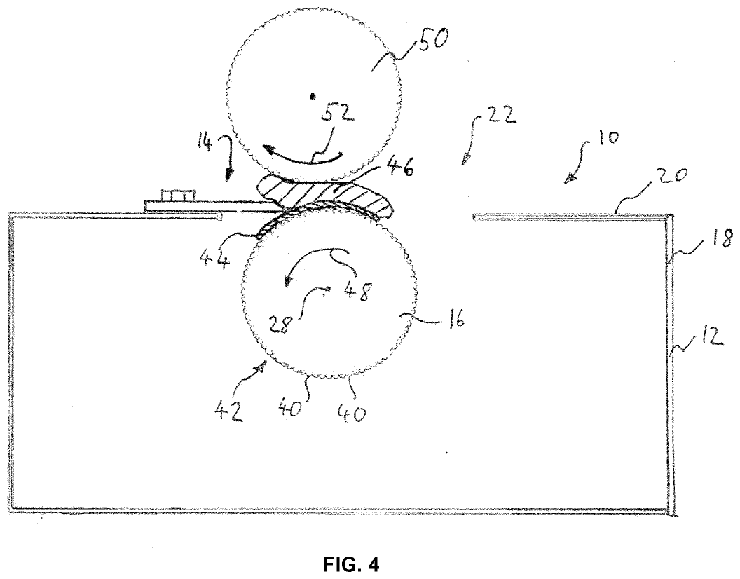

FIG. 4 shows an alternative embodiment of the device 10 in a view corresponding to FIGS. 2 and 3. The embodiment according to FIG. 4 differs from the embodiment from FIGS. 1 to 3 in that it comprises a pressure roller 5.

The pressure roller 50 is arranged on a side of the cutting edge 32 opposite the drawing roller 16. The pressure roller 50 is designed and arranged to press the full-thickness skin transplant 44 including the subcutaneous fatty tissue 46 against the drawing roller 16, meaning to press against it. This only means a light, directional pressing in the sense of a guiding.

Advantageously, the distance between drawing roller 16 and pressure roller 50 can be varied. It is preferred if the variation of the distance between drawing roller 16 and pressure roller 50 can be made via a displacement or a shifting of the pressure roller 50.

During operation of the device 10 according to the embodiment from FIG. 4, the full-thickness skin transplant 44 including the subcutaneous fatty tissue 46 is guided between pressure roller 50 and drawing roller 16. The drawing roller 16 is then set into motion via the hand crank 26. The pressure roller 50 is passively mounted. This means that the pressure roller 50 is not driven, but is only set into motion by contact with the subcutaneous fatty tissue 46, which is indicated by an arrow with the reference character 52. However, it is also conceivable that the rotation of the drawing roller 50 is coupled to the rotation of the drawing roller 16, for example mechanically. As a result, the surface speeds of the drawing roller 16 and the pressure roller 50 are preferably the same in terms of magnitude.

Because the full-thickness skin transplant 44 and the subcutaneous fatty tissue 46 are somewhat clamped between pressure roller 50 and drawing roller 16, they are guided by the rotation of the drawing roller 16 against the cutting edge 32 of the cutting unit 14, wherein the distance between drawing roller 16 and cutting edge 32 corresponds to the gap width 38 so that the cutting edge 32 sits precisely between fatty tissue 46 and full-thickness skin transplant 44 and separates them from one another.

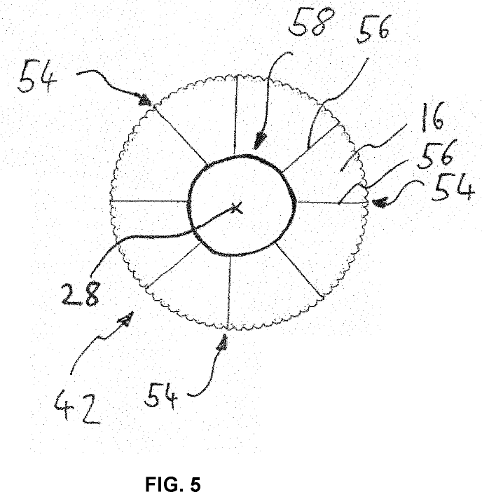

FIG. 5 shows an individual illustration of a drawing roller 16 of an alternative embodiment in a view corresponding to FIGS. 2 to 4. The drawing roller 16 of this embodiment has on its active surface 42 a plurality of suction openings 54 distributed over the active surface 42. The suction openings 54 are each connected to a vacuum channel 58 that extends along the axis of rotation 28 via channels 56.

The drawing roller 16 of this embodiment can be connected to a vacuum source (not depicted) via the vacuum channel 58. In this way, the suction openings 54 can be supplied with a vacuum so that they suction a full-thickness skin transplant 44 which is placed on the drawing roller 16 of this embodiment and hold it in close contact with the active surface 42 of the drawing roller 16.

Suction openings 54 of this type can also be provided on the pressure roller 50, wherein they then serve to suction the layer of subcutaneous fatty tissue 46 against the suction roller 50. By the provision of suction openings 54 in the drawing roller 16 as well as in the pressure roller 50, the separation effect of the cutting edge 32 can be increased.

It is also conceivable that the individual channels 56 are not connected to a central vacuum channel 58, but can each be connected individually to a vacuum source. In other words, in this embodiment, the channels 56 do not connect fluidically to each other.

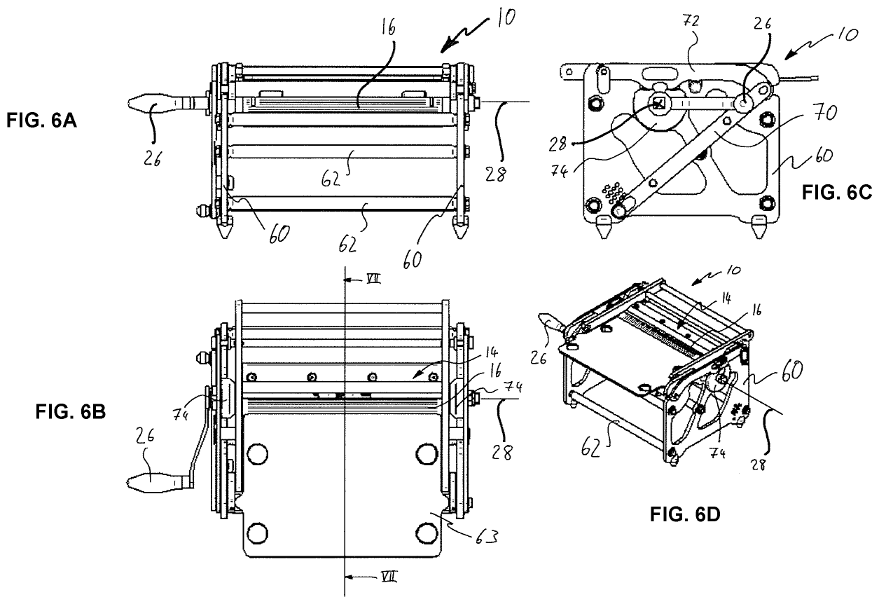

FIG. 6 shows an alternative embodiment of the device 10 according to the invention in different views (FIG. 6a in a front view; FIG. 6b in a top view; FIG. 6c in a side view and FIG. 6d in a perspective view).

The device 10 from FIG. 6 comprises a frame-like base body 12, the cutting unit 14 and the drawing roller 16.

The base body 12 in the present example comprises two plate-like side elements 60 that are connected to each other via beam-like longitudinal elements 62. The longitudinal elements extend parallel to the axis of rotation 28 of the drawing roller 16 and the side elements 60 are perpendicular to it.

The device 10 from FIG. 6 comprises an optional feed table 63 that is arranged opposite the cutting unit 14. The feed table 63 is designed for supporting the full-thickness skin transplant 44 with the subcutaneous fatty layer 46 located thereon. Via the feed table 63, the full-thickness skin transplant 44 can be fed to the drawing roller 16 via the feed table 63. Looking along the axis of rotation 28, the drawing roller 16 is arranged between the feed table 63 and the cutting edge 32.

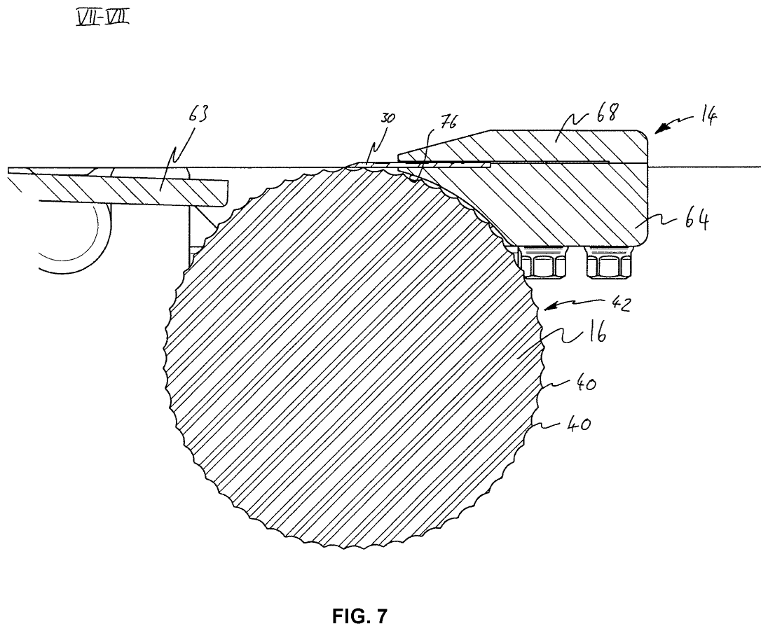

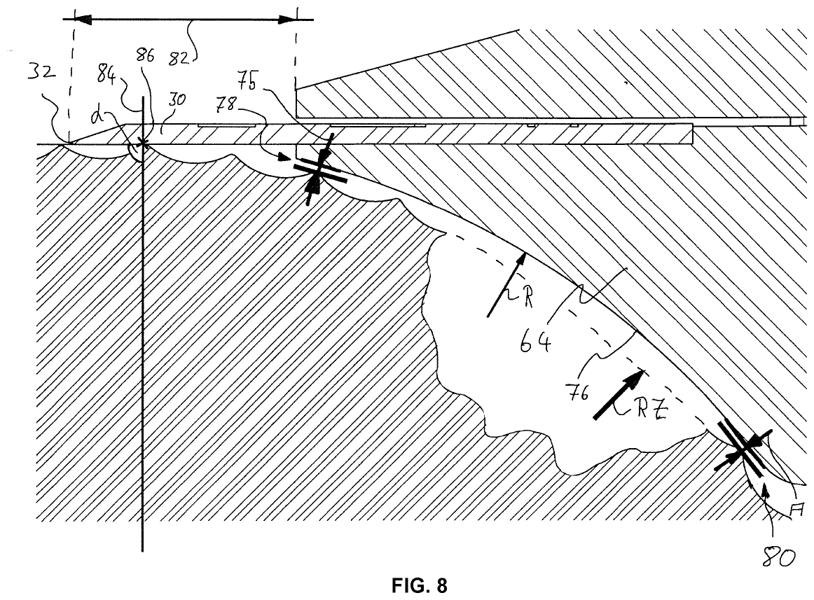

The cutting unit 14 of the device 10 from FIG. 6 is shown in detail in FIGS. 7 and 8 in a sectional view along the line VII-VII of the FIG. 6b. In FIG. 8, the drawing roller 16 is shown with a cutaway.

The cutting unit 14 comprises a blade holder 64, the blade 30 and a clamping element 68. The blade 30 comprises the cutting edge 32.

The blade 30 is releasably mounted in a play-free manner to the blade holder 64. In the present case, the mounting is realized via a clamping of the blade 30 between the blade holder 64 and the clamping element 68; the blade is braced in the blade holder 64 as a result. Other types of mounting are conceivable.

The blade holder 64 is arranged without play with respect to the drawing roller 16. The blade holder 64 and, thus, the cutting edge 32 can be adjustable in their position via a positioning lever 70, in particular in their spacing with respect to the drawing roller 16. The blade holder 64 and the drawing roller 16 can be locked or fixed in their respective position via a locking element 72. The drawing roller 16 is rotatably mounted with respect to the side elements 60 via sliding bearing 74 made from a polymer material, preferably PEEK (polyether ether ketone)(other polymer materials are conceivable).

The blade holder 64 has a surface 76 facing the drawing roller 16 that has a curved design. The surface 76 of the blade holder 64 that faces the drawing roller 16 is designed with a radius of curvature R which is larger than a radius of curvature RZ of the drawing roller 16. The blade holder 64 is preferably designed in such a manner and arranged with respect to the drawing roller 16 so that a secondary gap 75 that corresponds to the distance between the drawing roller 16 and the surface 76 of the blade holder 64 facing the drawing roller 16 on a side 78 of the blade holder close to the blade is less than a distance A on a side 80 of the blade holder 64 facing away from the blade holder. An advantageous transport behavior of the drawing roller 16 results from this. The secondary gap 75 in the example shown here is larger than the gap 34. The gap 34 in the present illustration is adjusted to 0. The secondary gap 75 or the distance between the drawing roller 16 and the surface 76 of the blade holder 64 facing the drawing roller 16 preferably increases with the increase of the gap 34 along the same extension of the surface 76.

The distance between the drawing roller 16 and the surface 76 of the blade holder 64 facing the drawing roller 16 is preferably designed so that it increases in the transport direction, meaning the direction of rotation of the drawing roller 16 during operation of the device 10.

The blade 30 preferably has a projection 82 over the blade holder 64 in such a manner that the cutting edge 32 is spaced between 2 mm and 6 mm, preferably between 3 mm and 5 mm from the blade holder 64.

The blade 30 is preferably designed flat and arranged with respect to the drawing roller 16 in such a manner that, looking along the axis of rotation 28 of the drawing roller 16 (illustration from FIGS. 7 and 8), a line 84 running perpendicular to the axis of rotation 28 cuts the blade 30 at an angle a of 90°, wherein the cutting edge 32 is preferably spaced at least 0.5 mm, preferably 1 mm, in particular 1.5 mm from the intersection 86 of the line 84 with the blade 30.

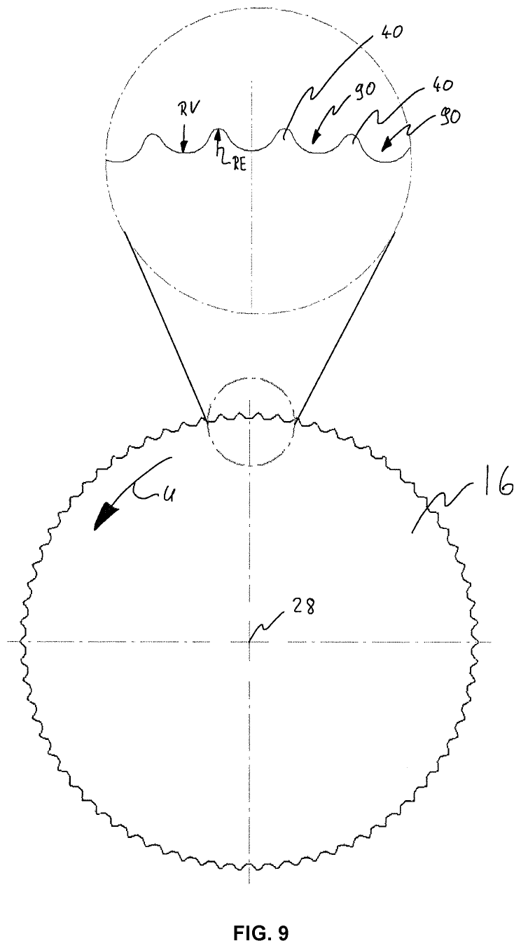

FIG. 9 shows an alternative embodiment of the drawing roller 16 in a section perpendicular to the axis of rotation 28. The drawing roller 16 has the protrusions 40 that are spaced apart from each other via recesses 90. The recesses 90 are designed with an arcuate cross-section in the present example. The recesses 90, as shown in FIG. 9, can have a radius of curvature RV that is larger than a radius of curvature RE of the protrusions. In the present example, the protrusions 40 are spaced further apart from each other in the circumferential direction U via recesses 90 than they are extended in the circumferential direction U.

What is claimed is

1. A device for degreasing a full-thickness skin transplant, comprising a cutting unit, which is designed and arranged to separate a layer of subcutaneous fatty tissue located on the full-thickness skin transplant, characterized in that the device comprises a drawing roller which is arranged across a gap at a distance from a cutting edge of the cutting unit, and which is designed to deliver the full-thickness skin transplant to the cutting unit by rotating the drawing roller.

2. The device according to claim 1, characterized in that the drawing roller can be rotated via a hand crank.

3. The device according to claim 1, characterized in that the drawing roller can be rotatably driven via a drive unit, wherein a rotational speed of the drawing roller is variable via a control element during the operation of the device.

4. The device according to claim 1, characterized in that a cutting edge of the cutting unit is arranged essentially parallel to an axis of rotation of the drawing roller.

5. The device according to claim 1, characterized in that the gap between the cutting edge of the cutting unit and the drawing roller has a gap width of 0.8-1.2 mm.

6. The device according to claim 1, characterized in that the drawing roller has rib-like protrusions on its active surface, wherein the rib-like protrusions run parallel to an axis of rotation of the drawing roller.

7. The device according to claim 1, characterized in that a preferably flexible pressure roller is arranged on a side of the cutting edge opposite the drawing roller that is designed to press the full-skin transplant with the subcutaneous fatty tissue located thereon against the drawing roller.

8. The device according to claim 1, characterized in that the gap width between the cutting edge and the drawing roller is adjustable.

9. The device according to claim 1, characterized in that the drawing roller has a rounded active surface at least in some regions and a rounded design over its entire active surface.

10. The device according to claim 1, characterized in that the drawing roller has at least one suction opening on its active surface.

11. The device according to claim 1, characterized in that the device has a feed table for supporting the full-thickness skin transplant with the subcutaneous fatty tissue located thereon, via which the full-thickness skin transplant can be fed to the drawing roller, wherein the drawing roller is arranged between the feed table and the cutting edge when looking along the axis of rotation.

12. The use of a device for degreasing a full-thickness skin transplant, wherein the device comprises a cutting unit, which is designed and arranged to separate a layer of subcutaneous fatty tissue located on the full-thickness skin transplant, characterized in that the device comprises a drawing roller which is arranged across a gap at a distance from a cutting edge of the cutting unit, and which is designed to deliver the full-thickness skin transplant to the cutting unit by rotating the drawing roller..

13. A method for degreasing a full-thickness skin transplant, wherein the method comprises the steps:
bringing the full-thickness skin transplant with the subcutaneous fatty tissue located thereon onto a drawing roller in such a manner that the full-thickness skin transplant faces the drawing roller and the subcutaneous fatty tissue faces away from the drawing roller;
guiding the full-thickness skin transplant with the subcutaneous fatty tissue located thereon by rotation of the drawing roller against a cutting edge of a cutting unit, wherein a gap between the drawing roller and the cutting unit is designed in such a manner that the full-thickness skin transplant is guided by rotation of the drawing roller between the drawing roller and the cutting unit into the gap, while the cutting unit separates the subcutaneous fatty tissue from the full-thickness skin transplant.

14. The method according to claim 13, characterized in that the rotation of the drawing roller is accomplished manually via a hand crank.

15. The method according to claim 13, characterized in that it is carried out using a device comprising a cutting unit, which is designed and arranged to separate a layer of subcutaneous fatty tissue located on the full-thickness skin transplant, characterized in that the device comprises a drawing roller which is arranged across a gap at a distance from a cutting edge of the cutting unit, and which is designed to deliver the full-thickness skin transplant to the cutting unit by rotating the drawing roller. comprising a cutting unit, which is designed and arranged to separate a layer of subcutaneous fatty tissue located on the full-thickness skin transplant, characterized in that the device comprises a drawing roller which is arranged across a gap at a distance from a cutting edge of the cutting unit, and which is designed to deliver the full-thickness skin transplant to the cutting unit by rotating the drawing roller.

16. The device according to claim 10, characterized in that the drawing roller plurality of suction openings distributed over the active surface that can be connected with a vacuum source.
